(19) United States
(12) Patent Application Publication
(10) Pub. No.: US 2016/0017370 A1
(43) Pub. Date: Jan. 21, 2016

(54) DEVICE FOR INTRACELLULAR DELIVERY AND A METHOD THEREOF

(71) Applicant: City University of Hong Kong, Kowloon (HK)

(72) Inventors: Peng Shi, Kowloon (HK); Xianfeng Chen, New Territories (HK); Wenjun Zhang, New Territories (HK)

(21) Appl. No.: 14/719,416

(22) Filed: May 22, 2015

(30) Foreign Application Priority Data
Jul. 15, 2014 (CN) 201410337234.5

(51) Int. Cl. C12N 15/89; C12M 1/42; C12N 5/00

(52) U.S. Cl. CPC C12N 15/89; C12N 5/0068; C12M 35/04

(57) ABSTRACT

A device for intracellular delivery includes a substrate having a diamond layer, and diamond nanoneedles that spaced apart from each other on the diamond layer, the substrate further comprises a silicon layer below the diamond layer, wherein the nanoneedles are cylindrical nanoneedles, and a side surface of the nanoneedles is perpendicular to the diamond layer.

### CROSS-REFERENCE TO RELATED APPLICATIONS

The present application claims priority to Chinese Patent Application No. 201420337234.5, filed Jul. 15, 2014, and incorporated herein by reference.

### FIELD OF THE INVENTION

The present invention is related to the field of biotechnology, particular but not exclusively, related to a device and a method for intracellular delivery, and a method for preparing a device for intracellular delivery.

### BACKGROUND OF THE INVENTION

Efficient delivery of molecules and materials into living cells is a very important topic in cellular biotechnology. It is of great value to basic study of cell biology, development of drugs and clinical treatments. For instance, reprogramming somatic cells to an induced pluripotent-stem-cell (iPS) state can be achieved by intracellular delivery of genes, proteins, or mRNA of specific transcriptional factors, which holds the potential to revolutionize regenerative medicine. Numerous other materials such as siRNA, peptides and nanoparticles are also potential candidates for medical applications.

Many strategies have been developed to facilitate the cross-membrane movement of molecules. Each established method has its own advantages and drawbacks regarding different aspects of the delivery process, including efficiency, expression level, toxicity cell viability, and equipment requirements. For instance, viral vector based techniques are limited to nuclide acid delivery, and the procedures are labor-intensive, often involving various safety issues. Although chemical methods such as lipofection is relatively simple to perform, the efficiency for post-mitotic cells are typically very low (around 1 to 2% in neuron), and is not suitable for protein, nanomaterials or the like.

Calcium phosphate precipitation is a cost-effective method, but it is difficult to yield reproducible results and the transfection efficiency is also low. Electrical methods temporarily alter the properties of cell membranes by exposing them to voltage pulses to allow charged materials to enter cells. However, they usually require cells in suspension and the toxicity can vary dramatically depending on different cell types.

Mechanical disruption to cell membranes is emerging as a promising method for intracellular delivery. For example, single nanoneedle with a diameter below 800 nm has been used for intracellular delivery without causing serious damage to cells. However, this approach requires the use of atomic force microscope (AFM), and the throughput is extremely low. Even though arrays of carbon nanofibers or nanoneedles were applied to improve the efficiency, cells are still required to be suspended and the nanoneedles are required to be modified with material prepared for delivery. Also, complicated devices are involved in these existing methods. The existing methods and devices cannot be applied in adherent cells or non-mitotic cells such as neurons in primary culture. Accordingly, there remains a need for developing a better device and method for intracellular delivery.

### SUMMARY OF THE INVENTION

The present invention relates to a device for intracellular delivery and a method thereof with unexpected results. The method for intracellular delivery attains a significant improvement in delivery efficiency and cell viability when compared with the existing technologies.

According to a first aspect of the present invention, there is provided a device for intracellular delivery, comprising a substrate having a diamond layer, and diamond nanoneedles that spaced apart from each other on the diamond layer, the substrate further comprises a silicon layer below the diamond layer, wherein the nanoneedles are cylindrical nanoneedles, and a side surface of the nanoneedles is perpendicular to the diamond layer.

According to a second aspect of the present invention, there is provided a method for intracellular delivery, comprising the steps of: a) depositing a cell in a culture medium on a plate, the culture medium comprises a material to be delivered; b) providing a device for intracellular delivery on a liquid surface of the culture medium to form a sandwich structure, the device for intracellular delivery includes a substrate and nanoneedles that attached on a substrate surface and spaced apart from each other, the nanoneedles are made from diamond; tips of the nanoneedles point towards the cells; c) centrifuging the sandwich structure at a centrifugation condition that allows the tips of the nanoneedles to pierce the cells.

According to a third aspect of the present invention, there is provided a method of preparing a device for intracellular delivery, characterized in that: the device comprise a substrate having a diamond layer, and diamond nanoneedles formed on the diamond layer, the diamond nanoneedles spaced apart from each other, the substrate further comprises a silicon layer below the diamond layer, characterized in that: the nanoneedles are cylindrical nanoneedles, and a side surface of the nanoneedles is perpendicular to the diamond layer; the method further comprises the following steps: a) forming a nanodiamond film on the silicon layer, a deposition condition allows the nanodiamond film to have a thickness of 0.5-5 μm larger than a desired height of the nanoneedles; b) performing a bias-assisted reactive ion etching on the nanodiamond film formed, wherein the bias-assisted reactive ion etching is performed under the conditions of: a reactive pressure of 4×10-3 to 8×10-3 Torr, a reaction time from 20 minutes to 4 hours; a bias pressure of −50V to −250V; and a gas for bias-assisted reactive ion etching is selected from the group consisting: H₂, a mixed gas of Ar and H₂, and a mixed gas of CH₄ and H₂, and a combination thereof.

According to a fourth aspect of the present invention, there is provided a method for disrupting a cell membrane, comprising the steps of: providing a centrifuge; providing a cell incubated in a container with a medium, the medium comprising a liquid surface; placing a device having nanoneedles on a liquid surface of the medium to form a sandwich structure; centrifuging the sandwich structure with a centrifugal force; wherein the nanoneedles are cylindrical, and the centrifugation force is applied with an acceleration rate of from about 0.001 to about 0.003 g/s, and a deceleration rate of about 0.003 to about 0.006 g/s.

### BRIEF DESCRIPTION OF THE DRAWING(S)

Embodiments of the present invention will now be described, by way of example, with reference to the accompanying drawings in which:

FIG. 1 is a Scanning Electron Micrograph (SEM) of a device according to an embodiment of the present invention.

FIG. 2 is a partially enlarged SEM of FIG. 1, showing a device according to an embodiment of the present invention.

FIG. 3 is a partially enlarged SEM of a device according to the prior art.

FIGS. 4a, 4b, 4c, and 4d together show fluorescence images of primary neurons expressing markers (MAP2 and vGlut1) after successful delivery of GFP plasmid DNA into the primary neurons by a device according to an embodiment of the present invention.

FIG. 5 is a schematic diagram showing a method of a preferred embodiment of the present invention.

### DETAILED DESCRIPTION OF EMBODIMENTS OF THE INVENTION

In the present invention, the term “on” or “below” used for describing the device of the present invention is considered with reference to the direction of the height of the nanoneedles, unless otherwise indicated. For the method for intracellular delivery, the term “on” or “below” used should be referred to the direction of the gravity.

The present invention provides a device for intracellular delivery. The device includes a substrate having a diamond layer, and diamond nanoneedles that spaced apart from each other on the diamond layer, the substrate further includes a silicon layer below the diamond layer, wherein the nanoneedles are cylindrical nanoneedles, and a side surface of the nanoneedles is perpendicular to the diamond layer.

Preferably, the nanoneedles of the device of the present invention pierce cells for delivering a material into the cells through the pierced holes. The nanoneedles stand upright on the diamond layer to form a nanoneedle array.

The term “cylindrical” further includes a cylindrical shape deformed to a certain extent. For instance, in a direction parallel to the diamond layer, variations of the cross-section of the nanoneedle and the maximum distance between any two points on the cross-section may not exceed 10%. Alternatively, in a direction parallel to the diamond layer, the cross-section of the nanoneedles may have a nearly circular shape. For example, the ratio of maximum distance to minimum distance between any two points on an edge of the cross-section may be from about 1:1 to about 1.3:1.

The term “perpendicular to the diamond layer” further includes the embodiments where the nanoneedles are nearly perpendicular to the diamond layer. The side surface of the nanoneedles may form an angle of 85 to 95 degrees with the diamond layer. Preferably, the cylindrical nanoneedles have a vertical side wall to achieve a better piercing performance. Without intending to be limited by theory, it is believed that the nanoneedle having a vertical wall can perform better with a more consistent piercing effect when compared with the cone-shaped nanoneedle having a tapering wall.

In the present invention, the nanoneedles have a diameter that used for intracellular delivery. For example, the diameter is about 10-800 nm and preferably about 50-600 nm. The more preferable diameter is about 200-450 nm so as to further enhance the intracellular delivery efficiency. The term “diameter” refers to the extent of the cross-section of the nanoneedle on a direction parallel to the diamond layer.

The nanoneedles have a height that used for intracellular delivery, for example, the height is about 3-8 μm and preferably about 3.5-6.5 μm. The more preferable height is about 3.8-5.3 μm so as to further enhance the intracellular delivery efficiency.

In one embodiment, the distribution density of the nanoneedles on the diamond layer may be the density of the nanoneedle array for intracellular delivery of a material. For example, the distribution density is about 1×10⁶ to about 15×10⁶ per cm². More preferably, the distribution density is about 4×10⁶ to about 8×10⁶ per cm². It is believed that the increase in density can enhance the delivery efficiency. However, too high of a density may also lead to an undesirable cell death. Accordingly, the most preferable distribution density is in a range of about 4×10⁶ to about 8×10⁶ per cm².

Preferably, the diamond layer has a thickness to be applied in a nanoneedles array for intracellular delivery of a material. For example, the thickness of the diamond layer is about 0.5-5 μm. More preferably, the thickness of the diamond layer is about 1-4 μm.

In the present invention, the silicon layer has a thickness to be applied in a nanoneedles array for intracellular delivery. For example, the thickness of the silicon layer is about 400-600 μm. More preferably, the thickness of the silicon layer is about 480-520 μm.

The present invention also provides a method for intracellular delivery. The method includes the steps of: (a) depositing cells in a culture medium on a plate, the culture medium includes a material to be delivered; b) providing a device for intracellular delivery on a liquid surface of the culture medium to form a sandwich structure, the device for intracellular delivery includes a substrate and nanoneedles that attached on a substrate surface and spaced apart from each other, the nanoneedles are made from diamond; tips of the nanoneedles point towards the cells; and c) centrifuging the sandwich structure at a centrifugation condition that allows the tips of the nanoneedles to pierce the cells.

Preferably, the cells are adhered on the plate for growth. The cells may also be suspended in the culture medium for growth, or transformed from adherent cells to suspension cells.

In one embodiment, the cells can be cultured in common growth medium. The growth medium can be replaced with a culture medium that contains a material to be delivered before performing the intracellular delivery. The culture medium that contains the material to be delivered can be prepared by adding the material to be delivered to the growth medium. The growth medium may be any commonly used culture medium for cell culture. For example, the growth medium may be selected from the group consisting of: DMEM culture medium, F-12 culture medium, 1640 culture medium and Neural Basal culture medium. Serum such as fetal bovine serum and/or calf serum may be introduced into the growth medium.

Preferably, the device for intracellular delivery is provided on the liquid surface of the culture medium. The device for intracellular delivery can float on the liquid surface of the culture medium due to the surface tension of the culture medium, or sink below the liquid surface to cover the cells.

Preferably, the device for intracellular delivery is the one that described in the present invention for intracellular delivery. Regarding the device for intracellular delivery, the nanoneedles are cylindrical and a side surface of the nanoneedles is perpendicular to the diamond layer.

Alternatively, the device for intracellular delivery is not limited to the above context. For instance, the nanoneedles of the device for intracellular delivery may be cone-shaped. The bottom surfaces of the cone-shaped nanoneedles are connected to the diamond layer. The nanoneedle may be a circular cone or a polygonal cone. The term “cone-shaped” indicates that the side surface of the nanoneedle may form an angle of about 85 to 95 degrees with the diamond layer. The cone-shaped nanoneedle may have a height of about 1-10 μm, and a bottom surface diameter of about 0.5-2 μm. Said diameter refers to the maximum distance between any two points on the cross-section of the nanoneedle on a direction parallel to the diamond layer.

The cone-shaped nanoneedle may be formed by connecting two separate parts. The lower part is a base portion while the upper part is a cone portion. The diameter of an upper end of the base portion is about 250-700 nm, and the diameter of a bottom end of the stage is about 1000-1900 nm. The base portion has a height of about 6-9 μm. The cone portion has a diameter of about 100-150 nm, and a height of about 300-500 nm. The nanoneedles are distributed on the diamond layer at a distribution density of about 0.5×10⁶/cm² to about 1.5×10⁶/cm².

The centrifugation condition allows the tips of the nanoneedles to pierce the cells. In a preferred embodiment, the centrifugation condition is: a relative centrifugation force of about 10-15 g, preferably about 12-13 g. The centrifugation may last for about 30-300 s, and preferably about 120-180 s.

The amount of culture medium containing the material to be delivered may be 20-250 μl per cm² of adherent cells.

The cells may be adherent cells or suspension cells. The source of the cells may be selected from the group consisting of: animal cells, bacterial cells, fungal cells, plant protoplasts, and a combination thereof. The cells may be selected from the group consisting of: primary cultured cells, sub-cultured cells, and a combination thereof. Preferably, the cells are NIH3T3 cells, primary hippocampal neural progenitor cells, fibroblasts and A549 cells.

In a preferred embodiment, the centrifugation involves an acceleration stage and a deceleration stage under a relatively gentle acceleration or deceleration condition. More preferably, the relative centrifugal force of the centrifugation during an acceleration stage has an acceleration rate of about 0.001-0.003 g/s; the relative centrifugal force of the centrifugation during a deceleration stage has a deceleration rate of about 0.003-0.006 g/s. It is believed that such gentle acceleration and deceleration ensure a smooth application of the device on the target cells. Such a gentle and smooth application avoids substantial cell death due to the vigorous movement of the device against the cells.

In one embodiment, the force (F) applied to each individual nanoneedle may be well-controlled according to the practical needs via adjusting different parameters in accordance with the following formula:

$$F = \frac{m {ω}^{2} r}{n} = \frac{{mg}^{′}}{n} = \frac{{ρ}_{si} {L}^{2} H \cdot {g}^{′}}{N \cdot {L}^{2}} .$$

Where L is the length of the nanoneedle, H is the thickness of the device, N is the distribution density of the nanoneedles, ρₛᵢ is density of silicon (for example, about 2.33×10³ kg/m³), n is the total number nanoneedles on the device, r is the length of spinning arm of the centrifuge, ω is the spinning speed, and g′ is the relative centrifugal force measured in multiples of earth gravity acceleration. Accordingly, the present invention provides an approach of well-controlling the force exerted on the individual target cell for intracellular delivery with a prominent cell viability.

Preferably, after the centrifugation, the culture medium that contains the material to be delivered may be introduced to float the device for delivery, so that the delivery efficiency is further improved. The amount of culture medium containing the material to be delivered, which used to float the device, may be about 250-650 μl per cm² of adherent cells.

Upon the removal of the device from the culture medium, the culture medium containing the material to be delivered is remained in the plate for 5-60 minutes to facilitate the delivery efficiency.

Preferably, after the above step, the culture medium containing the material to be delivered may be replaced with a fresh growth medium.

The material to be delivered may be any material that commonly used for intracellular delivery in the field of cell biotechnology. For example, the material to be delivered is selected from the group consisting of: a DNA, a RNA, a PNA, a dye, a protein, an antibody, a small molecule drug, a nanoparticle, and a combination thereof; wherein the dye may be selected from the group consisting of: ethidium homodimer, fluorescein isothiocyanate (FITC) labeled dextran, a quantum dot and a combination thereof. The nanoparticle may include a polyethylene nanoparticle. The DNA refers to a deoxyribonucleic acid, RNA refers to a ribonucleic acid, and PNA refers to a peptide nucleic acid.

The concentration of the material to be delivered within the culture medium may be any commonly used concentration in the field of cell biotechnology. For example, in the culture medium, DNA concentration may be about 0.5-2 μg/ml; antibody concentration may be about 0.5-2 μg/ml, ethidium homodimer concentration may be about 0.5-2 μg/ml; FITC labeled dextran concentration may be about 0.2-0.8 mg/ml; quantum dot concentration may be 1-40 nM; polyethylene nanoparticle concentration may be about 3×10⁻⁵ to about 5×10⁻⁵% (w/v).

In one preferred embodiment of the present invention, the material to be delivered is selected from the group consisting of: a DNA, a RNA, a PNA and a combination thereof. The culture medium further includes a transfection reagent, wherein the transfection reagent includes a cationic liposome. Preferably, the cationic liposome includes Lipofectamine, wherein the concentration of the cationic liposome may be about 0.5-2 μg/ml.

The method for intracellular delivery of the present invention may be applied on in vitro cells only and not on in vivo cells of the living animals. The treated in vitro cells may not be implanted into the living animals, and may not be cultured into a living object. Alternatively, it is possible that the treated in vitro cells may be implanted into the living animals, or may grow into a living object.

The present invention further provides a method for preparing a device for intracellular delivery. The device has a substrate having a diamond layer, and diamond nanoneedles that spaced apart from each other on the diamond layer, the substrate further has a silicon layer below the diamond layer, wherein the nanoneedles are cylindrical nanoneedles, and a side surface of the nanoneedles is perpendicular to the diamond layer.

The method for preparing the device includes the steps of: a) forming a nanodiamond film on the silicon layer, a deposition condition allows the nanodiamond film to have a thickness of about 0.5-5 μm larger than a desired height of the nanoneedles; b) performing a bias-assisted reactive ion etching on the nanodiamond film formed, wherein the bias-assisted reactive ion etching is performed under the conditions of: a reactive pressure of about 4×10⁻³ to about 8×10⁻³ Torr, a reaction time from about 20 minutes to about 4 hours; a bias pressure of about −50V to about −250V; a gas for bias-assisted reactive ion etching is selected from the group consisting of: H₂, a mixed gas of Ar and H₂, a mixed gas of CH₄ and H₂, and a combination thereof.

In one embodiment, the silicon layer may be a silicon wafer. The diameter of the silicon wafer may be about 1-20 cm, preferably about 5-10 cm. The thickness of the silicon wafer may be about 400-600 μm, preferably about 480-520 μm. The silicon wafer may be obtained from cutting of a monocrystalline silicon. The monocrystalline silicon may be an n-type monocrystalline silicon or a p-type monocrystalline.

The method may further include: polishing the silicon layer with a polishing agent before forming a nanodiamond film on the silicon layer; the polishing agent may include diamond nanoparticles and an organic solvent. The particle size of the diamond nanoparticles may be about 3-8 nm per 1 ml of the organic solvent, and the organic solvent may be ethanol. Preferably, the silicon layer is polished for about 30-90 minutes.

The step of forming the nanodiamond film on the silicon layer may be achieved by any known methods in the art, for example, using microwave plasma chemical vapor deposition (MPCVD). In one embodiment, the commercially available MPCVD system and apparatus (ASTeX) were used. The MPCVD is performed under the condition of: 0.8-1.6 kW microwave power; a mixed gas of CH₄ and H₂ as a plasma-inducing gas, wherein the volume ratio of CH₄ and H₂ may be about 0.05:1 to about 0.2:1; the gas pressure for deposition of about 20-40 Torr, the speed of the gas flow of 100-300 sccm; and the temperature of about 700-900° C. The thickness of the resultant nanodiamond film can be controlled by varying the duration of the deposition. For instance, it takes about 15-20 hours to deposit a nanodiamond film with a thickness of about 7-10 μm.

In one embodiment, the bias-assisted reactive ion etching (bias-assisted RIE) of the nanodiamond film may be performed by electron cyclotron resonance microwave plasma chemical vapor deposition (ECR MPCVD) or other known methods in the field. In this embodiment, a commercial MPCVD apparatus equipped with a microwave source (ASTeX) was used. The microwave source employs an external magnetic field of 800-950 Gauss generated by an external magnetic coil. The bias-assisted reaction ion etching is performed under the condition of: a reactive pressure of about 4×10⁻³ to about 8×10⁻³ Torr, a reaction time from about 20 minutes to about 4 hours; a bias pressure of about −50V to about −250V; and a gas for bias-assisted reactive ion etching being selected from the group consisting of: H₂, a mixed gas of Ar and H₂, and a mixed gas of CH₄ and H₂, and a combination thereof.

Since the reactive pressure and the reaction time are under controlled, the nanoneedles of the device for intracellular delivery prepared are substantially perpendicular to the diamond layer. In a further embodiment, the bias-assisted reactive ion etching may be performed under the condition of: the flow rate of the gas being about 10-30 sccm, and the microwave power for the etching being about 0.4-1.2 kW.

The device for intracellular delivery may be adjusted or chopped into a suitable size for individual need. For example, a well of a 24-well culture plate may have a diameter of 15 mm and therefore the device may be cut into a shape having a diameter of about 10-14 mm for use.

The present invention is further described with the following examples.

### Example 1

This example exemplifies a device for intracellular delivery and the method thereof of the present invention.

An n-type monocrystalline silicon wafer with a diameter of 7.5 cm and a thickness of 500 μm was ultrasonically abraded for 60 minutes by using a polishing agent. The polishing agent was a suspension of nanodiamond particles with a particle size of 5 nm in ethanol.

The abraded silicon wafer was placed into a MPCVD apparatus (ASTeX), for preforming the nanodiamond layer deposition. The deposition condition was: a microwave power of 1.2 kW; a mixed gas CH₄ and H₂ as a plasma-inducing gas, wherein the volume ratio of CH₄ and H₂ was 0.11:1; the gas pressure for the deposition of 30 Torr and the gas flow rate of 200 sccm; and the temperature of about 800° C. It took 15 hours to deposit a nanodiamond film with a thickness of 7 μm. After the deposition, a nanodiamond film was formed on the silicon wafer. The thickness of the nanodiamond film had been confirmed to be 7 μm using a scanning electron microscope.

A bias-assisted RIE was performed on the freshly formed nanodiamond layer by using a commercial MPCVD apparatus equipped with a microwave source (ASTeX). The microwave source employed an external magnetic field of 875 Gauss generated by an external magnetic coil. The bias-assisted RIE was performed under the condition of: a reactive pressure of 7×10⁻³ Torr, a reaction time of 3 hours; H₂ as a reacting gas for bias-assisted reactive ion etching; a reacting gas flow rate of 20 sccm; a bias pressure of −200V; and a microwave power of 0.8 kW. The device for intracellular delivery of the present invention was thus obtained after the etching.

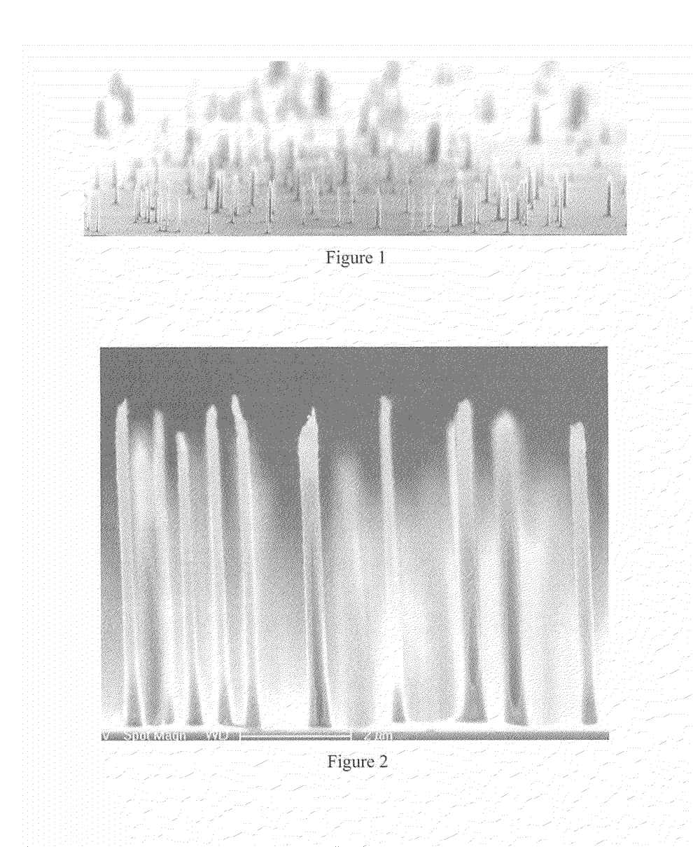

The morphology of the device for intracellular delivery was characterized by a scanning electron microscope (Philips, FEG SEM XL30). As shown in FIGS. 1 and 2, the device has the following features: the device has a substrate with a diamond layer, and diamond nanoneedles that spaced apart from each other on the diamond layer, the substrate further includes a silicon layer below the diamond layer, wherein the nanoneedles are cylindrical nanoneedles, and a side surface of the nanoneedles is perpendicular to the diamond layer. In a direction parallel to the diamond layer, variations of the cross-sections of the nanoneedle and the maximum distance between any two points on the cross-section do not exceed 10%. The side surface of the nanoneedles forms an angle of 85 to 95 degrees with the diamond layer. The nanoneedles have a diameter of 326±110 nm, and a height of 4.55±0.68 μm. The nanoneedles are distributed on the diamond layer with a distribution density of 6.66×10⁶/cm². After the etching, the diamond layer has a thickness of 1.2 μm.

### Reference Example 1

This reference example illustrated a device for intracellular delivery and the method thereof according to the prior art.

An n-type monocrystalline silicon wafer with a diameter of 7.5 cm and a thickness of 500 μm was ultrasonically abraded for 60 minutes by using a polishing agent. The polishing agent was a suspension of nanodiamond particles with a particle size of 5 nm in ethanol.

The abraded silicon wafer was placed into a MPCVD apparatus (ASTeX), for preforming the nanodiamond layer deposition. The deposition condition was: a microwave power of 1.2 kW; a mixed CH₄ and H₂ as a plasma-inducing gas, wherein the volume ratio of CH₄ and H₂ was 0.11:1; the gas pressure for the deposition of 30 Torr and the gas flow rate of 200 sccm; and the temperature of about 800° C. It took 16.5 hours to deposit a nanodiamond film with a thickness of 8 μm. After the deposition, a nanodiamond film was formed on the silicon wafer. The thickness of the nanodiamond film had been confirmed to be 8 μm using a scanning electron microscope.

A bias-assisted RIE was performed on the freshly formed nanodiamond layer by using a commercial MPCVD apparatus equipped with a microwave source (ASTeX). The microwave source employed an external magnetic field of about 875 Gauss generated by an external magnetic coil. The bias-assisted RIE was performed under the condition of: a reactive pressure of 6×10⁻³ Torr, a reaction time of 7 hours; H₂ as a reacting gas for bias-assisted reactive ion etching; a reacting gas flow rate of 20 sccm; a bias pressure of −200V; and a microwave power of 0.8 kW. The device for intracellular delivery of the prior art was thus obtained after the etching.

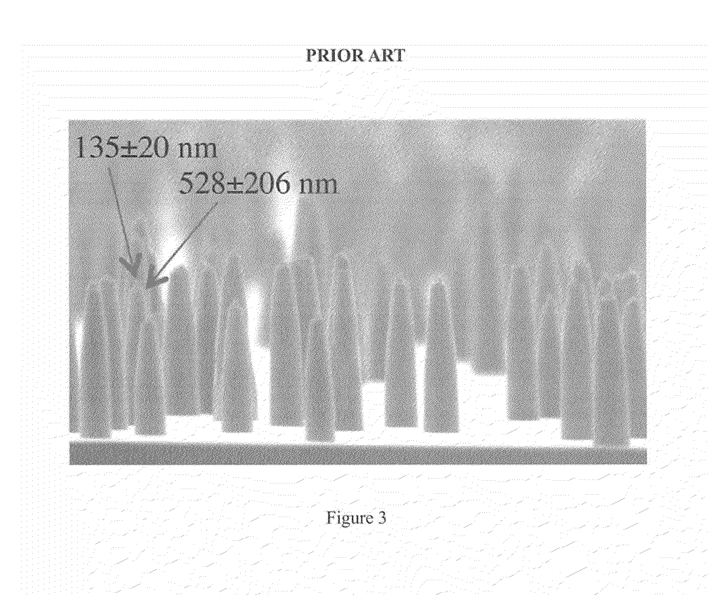

The morphology of the device for intracellular delivery was characterized by a scanning electron microscope (Philips, FEG SEM XL30). As shown in FIG. 3, the device has the following features: the device has a substrate with a diamond layer, and diamond nanoneedles that spaced apart from each other on the diamond layer, the substrate further includes a silicon layer below the diamond layer, wherein the nanoneedles are cone-shaped nanoneedles, and a side surface of the nanoneedles forms an angle of 65 to 75 degrees with the diamond layer. In a direction parallel to the diamond layer, variations of the cross-sections of the nanoneedle and the maximum distance between any two points on the cross-section exceed 25%. The nanoneedles were formed by two parts. The lower part is a base portion while the upper part is a cone portion. The diameter of an upper end of the base portion is 528±206 nm. The bottom end of the base portion has a diameter of 1600±310 nm. The base portion has a height of 7.42±1.35 μm. The cone portion has a diameter of 135±20 nm and a height of 413±103 nm.

The nanoneedles are distributed on the diamond layer with a distribution density of 1.1×10⁶/cm². After the etching, the diamond layer has a thickness of 1 μm.

### Example 2

This example exemplifies a device for intracellular delivery and the method thereof of the present invention.

An n-type monocrystalline silicon wafer with a diameter of 7.5 cm and a thickness of 400 μm was ultrasonically abraded for 60 minutes by using a polishing agent. The polishing agent is a suspension of nanodiamond particles with a particle size of 5 nm in ethanol.

The abraded silicon wafer was placed into a MPCVD apparatus, (ASTeX), for preforming the nanodiamond layer deposition. The deposition condition was: a microwave power of 0.8 kW; a mixed gas CH₄ and H₂ as a plasma-inducing gas, wherein the volume ratio of CH₄ and H₂ was 0.05:1; the gas pressure for the deposition of 20 Torr and the gas flow rate of 100 sccm; the temperature of about 700° C. It took 20 hours to deposit a nanodiamond film with a thickness of 10 μm. After the deposition, a nanodiamond film was formed on the silicon wafer. The thickness of the nanodiamond film had been confirmed to be 10 μm using a scanning electron microscope.

A bias-assisted RIE was performed on the freshly formed nanodiamond layer by using a commercial MPCVD apparatus equipped with a microwave source (ASTeX). The microwave source employed an external magnetic field of about 800 Gauss generated by an external magnetic coil. The bias-assisted RIE was performed under the condition of: a reactive pressure of 4×10⁻³ Torr, a reaction time of 20 minutes; Ar and H₂ were the reacting gases for bias-assisted reactive ion etching with a volume ratio of 0.4:1; a reacting gas flow rate of 20 sccm; a bias pressure of −50V; and a microwave power of 0.4 kW. The device for intracellular delivery of the present invention was thus obtained after the etching.

The morphology of the device for intracellular delivery was characterized by a scanning electron microscope (Philips, FEG SEM XL30). The device has the following features: the device has a substrate with a diamond layer, and diamond nanoneedles that spaced apart from each other on the diamond layer, the substrate further includes a silicon layer below the diamond layer, wherein the nanoneedles are cylindrical nanoneedles, and a side surface of the nanoneedles is perpendicular to the diamond layer. In a direction parallel to the diamond layer, variations of the cross-sections of the nanoneedle and the maximum distance between any two points on the cross-section do not exceed 10%. The side surface of the nanoneedles forms an angle of 85 to 95 degrees with the diamond layer. The nanoneedles have a diameter of 287±101 nm, and a height of 4.25±0.39 μm. The nanoneedles are distributed on the diamond layer with a distribution density of 4.8×10⁶/cm². After the etching, the diamond layer has a thickness of 3.7 μm.

### Example 3

This example exemplifies a device for intracellular delivery and the method thereof of the present invention.

An n-type monocrystalline silicon wafer with a diameter of 7.5 cm and a thickness of 600 μm was ultrasonically abraded for 60 minutes by using a polishing agent. The polishing agent was a suspension of nanodiamond particles with a particle size of 5 nm in ethanol.

The abraded silicon wafer was placed into a MPCVD apparatus (ASTeX), for preforming the nanodiamond layer deposition. The deposition condition was: a microwave power of 6 kW; a mixed gas CH₄ and H₂ as a plasma-inducing gas, wherein the volume ratio of CH₄ and H₂ was 0.18:1; the gas pressure for the deposition of 40 Torr and the gas flow rate of 300 sccm; the temperature of about 800° C. It took 18 hours to deposit a nanodiamond film with a thickness of 9 μm. After the deposition, a nanodiamond film was formed on the silicon wafer. The thickness of the nanodiamond film had been confirmed to be 7 μm using a scanning electron microscope.

A bias-assisted RIE was performed on the freshly formed nanodiamond layer by using a commercial MPCVD apparatus equipped with a microwave source (ASTeX). The microwave source employed an external magnetic field of about 950 Gauss generated by an external magnetic coil. The bias-assisted RIE was performed under the condition of: a reactive pressure of 8×10⁻³ Torr, a reaction time of 4 hours; a mixed gas of CH₄ and H₂ as a reacting gas for bias-assisted reactive ion etching with a volume ratio of 0.12:1; a reacting gas flow rate of 30 sccm; a bias pressure of −250V; and a microwave power of 1.2 kW. The device for intracellular delivery of the present invention was thus obtained after the etching.

The morphology of the device for intracellular delivery was characterized by a scanning electron microscope (Philips, FEG SEM XL30). The device has the following features: the device has a substrate with a diamond layer, and diamond nanoneedles that spaced apart from each other on the diamond layer, the substrate further includes a silicon layer below the diamond layer, wherein the nanoneedles are cylindrical nanoneedles, and a side surface of the nanoneedles is perpendicular to the diamond layer. In a direction parallel to the diamond layer, variations of the cross-sections of the nanoneedle and the maximum distance between any two points on the cross-section do not exceed 10%. The side surface of the nanoneedles forms an angle of 85 to 95 degrees with the diamond layer. The nanoneedles have a diameter of 327±123 nm, and a height of 4.57±0.59 μm. The nanoneedles are distributed on the diamond layer with a distribution density of 4.5×10⁶/cm². After the etching, the diamond layer has a thickness of 3.2 μm.

### Experiment 1

The devices for intracellular delivery prepared from the above Examples were used to illustrate the method for intracellular delivery of the present invention.

### Cell Culture

NIH3T3 fibroblasts and A549 cancer cells were cultured with a Dulbecco's modified eagle medium (DMEM medium, Life Technology) supplemented with L-glutamine, penicillin/streptomycin and 10% fetal bovine serum (FBS, HyClone). These cells were inoculated into a 4-well plate (Thermo Scientific) with the above medium before performing intracellular delivery.

For the primary neuron culture, hippocampal neurons were cultured on a 12 mm Germen coverslips (Bellco Glass). Before using, the coverslips were cleaned with 70 wt/wt concentrated nitric acid overnight and rinsed with sterile distilled water. The coverslips were further coated with 100 μg/ml polylysine (Sigma) overnight and coated with 10 μg/ml laminin for 4 hours before seeding neurons cells. Hippocampi tissue was dissected from E18 Sprague Dawley rats. Triturated enzymatic treated tissue was suspended in 1 ml DMEM solution containing 10% FBS and therefore a suspension of neurons was obtained. Neurons were then seeded onto the coated coverslips at a density of about 3×10⁴/cm² to about 5×10⁴/cm² in a 4-well plate. Two hours after the seeding, the neurons had completed the initial adhesion, and the medium was replaced by Neurobasal medium supplemented with B27, L-glutamine and penicillin/streptomycin. Half of the medium was replaced with fresh medium every 3-4 days.

### Material to be Delivered

The material to be delivered included: Calcein-AM (1 μM), ethidium homodimer-1 (EthD-1, 1 μM), 0.5 mg/ml FITC-labeled dextran (3 k-5 kDa, Sigma), 4×10⁻⁵% (w/v) polystyrene beads (200 nm, Wuhan Jiayuan), 1 μg/ml antibodies (Donkey IgG, Life Technology).

For delivery of quantum dots (QDs, Wuhan Jiayuan), different concentrations including 1.6 nM, 8 nM and 40 nM were tested in neurons. The water soluble QD has a CdSe/ZnS based core/shell structure with a 625 nm emission wavelength, and were modified with a layer of polyethylene glycol (PEG). To deliver green fluorescent protein (GFP) plasmid DNA, 1 μg/ml DNA was used and Lipofectamine 2000 (Life Technology) was added until the concentration became 1 ul/ml for acting as a transection agent.

### Intracellular Delivery

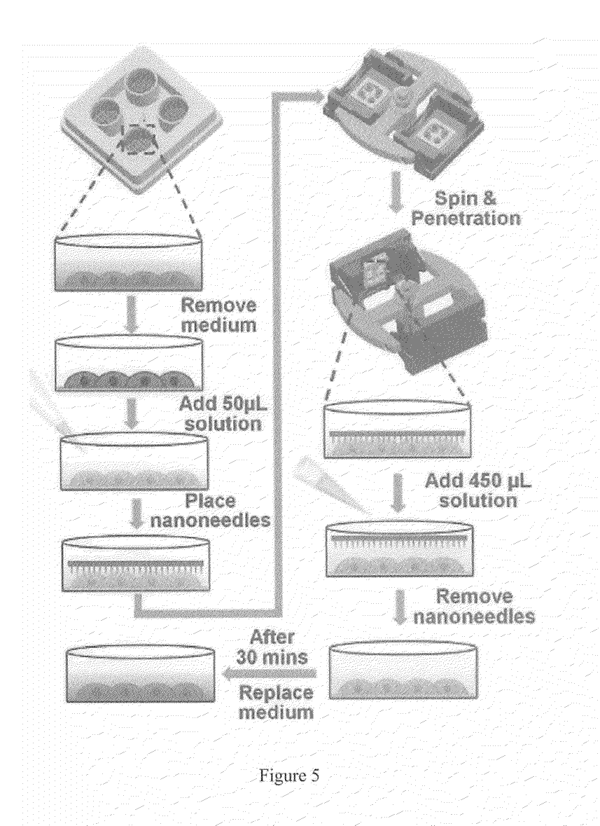

With reference to FIG. 5, to perform intracellular delivery of material into adherent cultured cells which incubated in a 4-well plate (with a diameter of 15 mm), the growth medium was firstly replaced with 50 μl culture medium containing material to be delivered such as fluorescent dye, dextran, fluorescent-labeled antibody, nanoparticle, DNA etc. Each of the devices for intracellular delivery as prepared in Examples 1 to 3 and Reference 1 was then placed onto the liquid surface of the culture medium containing the material to be delivered. As such, the pointed ends of the nanoneedles faced toward the cells to form a sandwich structure. In this embodiment, the sandwich structure consists of a culturing plate/dish, adherent cells, a culture medium containing the material to be delivered and the device for intracellular delivery from the bottom to the top.

The sandwich structure was placed in a centrifuge (Sorvall ST 16R, Thermo Scientific) with a plate rotor (M-20 microplate swinging bucket rotor, Thermo Scientific) and spun at various well-controlled speeds. The centrifugation was ramped at an acceleration rate of 0.002 g/s with respect to the relative centrifugal force at the acceleration stage so as to achieve a gentle acceleration. The acceleration was continued until the relative centrifugal force reached 12.5 g and then held at the spinning speed for 30 s before stating a gentle deceleration. The deceleration rate is 0.004 g/s with respect to the relative centrifugal force during the deceleration stage.

After centrifugation, 450 μl culture medium containing material to be delivered was added to float the device so that the device can thus be removed from the plate. The cells were further incubated in the culture medium for 30 minute. Fresh culture medium was added and replaced the culture medium that contained the material to be delivered. Accordingly, the intracellular delivery of the material was completed.

After the completion of the intracellular delivery, the cells were incubated and further studies might be conducted to investigate the delivery efficiency. The device for intracellular delivery was cleaned with a detergent with 98 wt/wt sulphuric acid. The amount of the detergent used is 1 ml per cm² of the diamond layer.

The delivery efficiency of EthD-1 and cell viability with respect to different device prepared in Examples 1 to 3 and Reference Example 1 were measured respectively based on the method of Decherchia (Decherchia, P., et al. Dual staining assessment of Schwann cell viability within whole peripheral nerves using calcein-AM and ethidium homodimer. J. Neurosci. Methods. 71, 205-213.1997). Table 1 shows the results.

TABLE 1

| | | Delivery efficiency (%) | | | | Cell viability (%) | | | |
|---|---|---|---|---|---|---|---|---|---|
| | | | | | Reference | | | | Reference |
| | Material | Example | | | Example | Example | | | Example |
| Cells | delivered | 1 | 2 | 3 | 1 | 1 | 2 | 3 | 1 |
| NIH | EthD-1 | 80.5 | 78.9 | 77.7 | 33.6 | 98.1 | 95.6 | 96.3 | 85.2 |
| 3T3 | | | | | | | | | |

The delivery efficiency of FITC labeled dextran and cell viability with respect to different device prepared in Examples 1 to 3 and Reference Example 1 were measured respectively based on the method of Sharei (Sharei, A. et al. A vector-free microfluidic platform for intracellular delivery. Proc. Natl Acad. Sci. USA 110, 2082-2087. 2013). Table 2 shows the results.

TABLE 2

| | | Delivery efficiency (%) | | | | Cell viability (%) | | | |
|---|---|---|---|---|---|---|---|---|---|
| | | | | | Reference | | | | Reference |
| | Material | Example | | | Example | Example | | | Example |
| Cells | delivered | 1 | 2 | 3 | 1 | 1 | 2 | 3 | 1 |
| NIH | FITC labeled | 64.5 | 58.1 | 57.2 | 23.6 | 97.1 | 96.6 | 95.3 | 83.2 |
| 3T3 | dextran | | | | | | | | |
| A549 | FITC labeled | 63.1 | 59.2 | 56.9 | 21.9 | 96.8 | 94.3 | 93.8 | 81.1 |
| | dextran | | | | | | | | |

The delivery efficiencies of EthD-1, FITC labeled dextran, fluorescent-labeled antibody (Donkey IgG), quantum dots, polyethylene nanoparticle and GFP plasmid DNAs, and cell viability with respect to different device prepared in Examples 1 to 3 and Reference Example 1 were measured respectively based on the method of Zeitelhofer (Zeitelhofer, M. et al. High-efficiency transfection of mammalian neurons via nucleofection. Nat. Protoc. 2, 1692-1704. 2007). Table 3 shows the results.

TABLE 3

| | | Delivery efficiency (%) | | | | Cell viability (%) | | | |
|---|---|---|---|---|---|---|---|---|---|
| | | | | | Reference | | | | Reference |
| | Material | Example | | | Example | Example | | | Example |
| Cells | delivered | 1 | 2 | 3 | 1 | 1 | 2 | 3 | 1 |
| Primary | EthD-1 | 80.2 | 77.5 | 76.8 | 15.3 | 87.3 | 85.2 | 85.9 | 73.2 |
| neurons | | | | | | | | | |
| Primary | FITC labeled | 62.5 | 59.7 | 55.3 | 13.9 | 86.9 | 85.7 | 85.3 | 71.0 |
| neurons | dextran | | | | | | | | |
| Primary | fluorescent- | 39.6 | 35.6 | 36.7 | 9.2 | 86.6 | 85.3 | 84.7 | 70.3 |
| neurons | labeled antibody | | | | | | | | |
| | (Donkey IgG) | | | | | | | | |
| Primary | quantum dots 1.6 nM | 61.9 | 59.3 | 58.7 | 14.8 | 87.1 | 85.9 | 84.4 | 69.8 |
| neurons | | | | | | | | | |
| Primary | quantum dots | 60.7 | 60.1 | 57.9 | 13.1 | 86.5 | 84.5 | 83.8 | 68.7 |
| neurons | 8 nM | | | | | | | | |
| Primary | quantum dots 40 nM | 59.9 | 61.3 | 58.2 | 12.9 | 86.2 | 87.6 | 84.1 | 69.5 |
| neurons | | | | | | | | | |
| Primary | polyethylene | 17.7 | 16.8 | 17.2 | 8.8 | 85.7 | 87.2 | 86.4 | 68.9 |
| neurons | nanoparticle | | | | | | | | |
| Primary | GFP plasmid | 49.6 | 48.7 | 47.6 | 10.3 | 84.2 | 83.6 | 82.9 | 65.3 |
| neurons | DNAs | | | | | | | | |

The results in Tables 1 to 3 show that the devices in Examples 1 to 3 and Reference Example 1 can perform the intracellular delivery of the present invention. Specifically, both of the delivery efficiency of material and cell viability obtained in Examples 1 to 3 are significantly higher than those of the Reference Example 1.

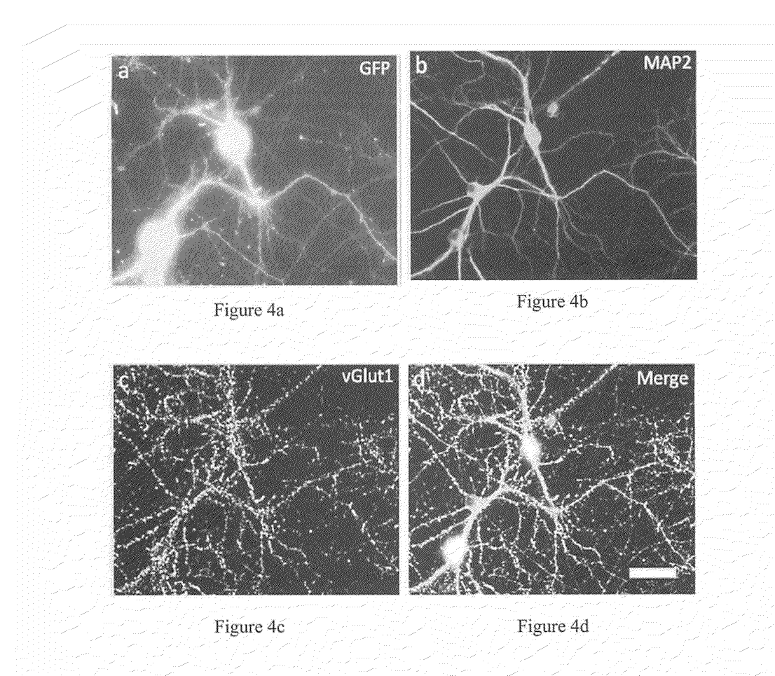

The characterized features (MAP2 and vGlut1) of the primary neurons after the intracellular delivery of GFP plasmid DNAs, performed by the device in Example 1, were further studied based on the method of Zeitelhofer (Zeitelhofer, M. et al. High-efficiency transfection of mammalian neurons via nucleofection. Nat. Protoc. 2, 1692-1704. 2007). MAP2 is Microtubule-associated protein 2 and vGlut1 is vesicular glutamine transporter 1. FIGS. 4a to 4d show the results and indicate that the primary neurons still had a good cell physiological activity after the intracellular delivery.

### Experiment 2

The device for intracellular delivery prepared from Example 1 was used to illustrate the method for intracellular delivery of the present invention, specifically under different centrifugal forces.

### Cell Culture

Hippocampal neurons were cultured on a 12 mm Germen coverslips (Bellco Glass). Before using, the coverslips were cleaned with 70 wt/wt concentrated nitric acid overnight and rinsed with sterile distilled water. The coverslips were further coated with 100 μg/ml polylysine (Sigma) overnight and coated with 10 μg/ml laminin for 4 hours before seeding neurons cells. Hippocampi tissue was dissected from E18 Sprague Dawley rats. Triturated enzymatic treated tissue was suspended in 1 ml DMEM solution containing 10% FBS and therefore a suspension of neurons was obtained. Neurons were then seeded onto the coated coverslips at a density of 3×10⁴/cm²-5×10⁴/cm² in a 4-well plate. Two hours after the seeding, the neurons had completed the initial adhesion, and the medium was replaced by Neurobasal medium supplemented with B27, L-glutamine and penicillin/streptomycin. Half of the medium was replaced with fresh medium every 3-4 days.

### Material to be Delivered

The material to be delivered was green fluorescent protein (GFP) plasmid DNA. 1 μg/ml DNA was used with or without Lipofectamine 2000 (Life Technology). Lipofectamine 2000 was added until the concentration became 1 ul/ml for acting as a transection agent.

### Intracellular Delivery

To perform intracellular delivery of material into adherent cultured cells incubated in a 4-well plate (with a diameter of 15 mm), the growth medium was firstly replaced with 50 μl culture medium containing material to be delivered, i.e. DNAs. The device for intracellular delivery as prepared in Example 1 was then placed onto the liquid surface of the culture medium containing the material to be delivered. As such, the pointed ends of the nanoneedles faced toward the cells to form a sandwich structure. In this embodiment, the sandwich structure consists of a culturing plate/dish, adherent cells, a culture medium containing the material to be delivered and the device for intracellular delivery from the bottom to the top.

The sandwich structure was placed in a centrifuge (Sorvall ST 16R, Thermo Scientific) with a plate rotor (M-20 microplate swinging bucket rotor, Thermo Scientific) and spun at various well-controlled speeds. The centrifugation was ramped at an acceleration rate of 0.002 g/s with respect to the relative centrifugal force at the acceleration stage so as to achieve a gentle acceleration. The acceleration was continued until the relative centrifugal force reached the desired value. The desired values include 8 g, 10, 12 g, 13 g, 15 g, 18 g and 30 g. Then, the spinning speed was held for 30 s before stating a gentle deceleration. The deceleration rate is 0.004 g/s with respect to the relative centrifugal force during the deceleration stage.

After centrifugation, 450 μl culture medium containing material to be delivered was added to float the device so that the device can thus be removed from the plate. The cells were further incubated in the culture medium for 30 minute. Fresh culture medium was added and replaced the culture medium that contained the material to be delivered. Accordingly, the intracellular delivery of the material was completed.

After the completion of the intracellular delivery, the cells were incubated for further studies to investigate the delivery efficiency.

The delivery efficiency of GFP plasmid DNAs and cell viability of primary neurons, using the device prepared in Example 1, were measured respectively based on the method of Zeitelhofer (Zeitelhofer, M. et al. High-efficiency transfection of mammalian neurons via nucleofection. Nat. Protoc. 2, 1692-1704. 2007). Table 4 shows the results.

TABLE 4

| | Relative centrifugal force (g) | | | | | | |
|---|---|---|---|---|---|---|---|
| | 8 | 10 | 12 | 13 | 15 | 18 | 30 |
| Delivery efficiency | 28.2 | 33.3 | 47.4 | 49.1 | 48.7 | 47.3 | 46.4 |
| (%) | | | | | | | |
| Cell viability (%) | 87.2 | 85.6 | 84.3 | 83.2 | 80.1 | 75.6 | 52.9 |

Table 4 shows that the delivery efficiency of the material and cell viability can be further enhanced when the relative centrifugal force is preferably about 10-15 g. More preferably, the relative centrifugal force is about 12 to 13 g.

### Experiment 3

The devices prepared from Examples 1 to 3 were used to illustrate that in addition to the method for intracellular delivery of the present invention, other possible methods may also be applied to perform the intracellular delivery. In turn, the device was affixed and the cells were ejected thereon for intracellular delivery. The results are compared with that obtained from using Reference Example 1.

### Cell Culture

A549 cancer cells were cultured with a Dulbecco's modified eagle medium (DMEM medium, Life Technology) supplemented with L-glutamine, penicillin/streptomycin and 10% fetal bovine serum (FBS, HyClone). These cells were inoculated into a 4-well plate (Thermo Scientific) with the above medium before performing intracellular delivery.

Hippocampal neurons were cultured on a 12 mm Germen coverslips (Bellco Glass). Before using, the coverslips were cleaned with 70 wt/wt concentrated nitric acid overnight and rinsed with sterile distilled water. The coverslips were further coated with 100 μg/ml polylysine (Sigma) overnight and coated with 10 μg/ml laminin for 4 hours before seeding neurons cells. Hippocampi tissue was dissected from E18 Sprague Dawley rats. Triturated enzymatic treated tissue was suspended in 1 ml DMEM solution containing 10% FBS and therefore a suspension of neurons was obtained. Neurons were then seeded onto the coated coverslips at a density of about 3×10⁴/cm² to about 5×10⁴/cm² in a 4-well plate. Two hours after the seeding, the neurons had completed the initial adhesion, and the medium was replaced by Neurobasal medium supplemented with B27, L-glutamine and penicillin/streptomycin. Half of the medium was replaced with fresh medium every 3-4 days.

### Material to be Delivered

The material to be delivered was green fluorescent protein (GFP) plasmid DNA, 1 μg/ml DNA was used and Lipofectamine 2000 (Life Technology) was added until the concentration became 1 ul/ml for acting as a transection agent.

### Intracellular Delivery

The devices prepared from Examples 1 to 3 and Reference Example 1 were placed into a 4-well plate with the nanoneedles pointing upwards.

Each of the A540 cancer cells and primary neurons was digested with trypsin and suspended at a cell concentration of 6×10⁴/ml with suitable growth medium. GFP plasmid DNAs and Lipofectamine 2000 were added into the cell suspension to obtain a cell suspension ready for intracellular delivery. The DNA concentration was 1 μg/ml and the Lipofectamine 2000 concentration was 1 μg/ml. A 1 ml pipette was used to obtain 1 ml of the cell suspension and eject the 1 ml cell suspension within 0.2 seconds to the device having nanoneedles facing upwards. The ejected cell suspension was collected and again ejected to the device for 9 more times. Accordingly, the intracellular delivery was completed. The cell suspension was finally transferred to a new 4-well plate for incubation. The delivery efficiency was further studied.

The delivery efficiency and cell viability with respect to different device prepared in Examples 1 to 3 and Reference Example 1 were measured respectively based on the methods of Chen (Chen, X. et al. A diamond nanoneedle array for potential high-throughput intracellular delivery. Adv. Healthc. Mater. 2, 1103-1107. 2013) and Sharei (Sharei, A. et al. A vector-free microfluidic platform for intracellular delivery. Proc. Natl Acad. Sci. USA 110, 2082-2087. 2013). Table 5 shows the results.

TABLE 5

| | | Delivery efficiency (%) | | | | Cell viability (%) | | | |
|---|---|---|---|---|---|---|---|---|---|
| | | | | | Reference | | | | Reference |
| | Material | Example | | | Example | Example | | | Example |
| Cells | delivered | 1 | 2 | 3 | 1 | 1 | 2 | 3 | 1 |
| A549 | GFP | 20.3 | 18.2 | 17.1 | 8.4 | 38.1 | 35.6 | 36.3 | 25.2 |
| Cancer | Plasmid | | | | | | | | |
| cells | DNAs | | | | | | | | |
| Primary | GFP | 1.6 | 1.5 | 2.1 | 1.1 | 2.3 | 4.3 | 3.8 | 2.0 |
| neurons | Plasmid | | | | | | | | |
| | DNAs | | | | | | | | |

Table 5 shows that all the devices in Examples 1 to 3 and Reference Example 1 can perform intracellular delivery on the mitotic cells (A549 cancer cells) in a way that the device is affixed the cells are ejected onto the device.

However, the delivery efficiency and the cell viability using such a method are lower than that achieved by using the method of the present invention (Experiment Examples 1 and 2). Nevertheless, the devices prepared from Examples 1 to 3 can achieve a better efficiency and cell viability when compared with that of Reference Example 1 in the same test. However, such a fixed device for piercing the cells ejected thereon may not be applied to treat the adherent cells when are not mitotic cells such as primary neurons for efficient intracellular delivery. This arrangement may also lead to a significant cell death in adherent non-mitotic cells.

As described herein, the device of the present invention is capable for piercing the cell membrane for intracellular delivery. Therefore, the present invention also provides a method of disrupting a cell membrane. The method includes the steps of: providing a centrifuge; providing a cell incubated in a container with a medium, the medium having a liquid surface; placing a device having nanoneedles on the liquid surface of the medium to form a sandwich structure; centrifuging the sandwich structure with a centrifugal force; wherein the nanoneedles are cylindrical, and the centrifugation force is applied with an acceleration rate of from about 0.001 to about 0.003 g/s, and a deceleration rate of about 0.003 to about 0.006 g/s.

The cell used herein can be adherent cell or suspension cell cultured or collected in a container such as a culture plate and a test tube. The device as described in the present invention may be placed on the liquid surface of the medium contained in the container so as to form a sandwich structure consisting of a container, a cell, a medium and a device. As such, when a centrifugal force is applied to the device, the nanoneedles pierce the cell membrane and cause a temporary cell membrane disruption.

Preferably, the cylindrical nanoneedles have a vertical side wall to achieve a better piercing performance. The vertical wall can produce a more consistent piercing effect on the cell membrane. As such, if the medium contains an additive to enter the cells via the pierced cell membrane, the additive may enter the individual cells in a more consistent rate so that it ensures most of the cells are delivered with a similar quantity of additive. The additive may be any material to be delivered into the cells such as a DNA, a RNA, a PNA, a dye, a protein, an antibody, a small molecule drug, a nanoparticle, and a combination thereof.

The centrifugal force applied on the cell may be adjusted according to the individual need. Preferably, the centrifugal force is applied with a gentle and smooth acceleration rate such that the forces acted on each of the nanoneedles are more consistent. The gentle deceleration rate also ensures that the forces acted on the nanoneedles are released steadily, but not vigorously which may further destroy the cells. Accordingly, the cell membranes of the cells are temporarily disrupted.

Alternatively, the entire centrifugation step may be repeated to ensure most of the cells have been pierced by the nanoneedles of the device, in particular for cell suspension.

Such a centrifugation-based method for disrupting of the cell membrane can achieve a precise control of the force applied through nanoneedles to disrupt the cell membrane. The precise control of the acceleration rate and deceleration rate further facilitate the piercing without causing significant cell death. This method may be useful in determining the cellular changes when there is a temporary cell membrane disruption, such as an electrolyte leakage. Without intending to be limited by theory, it is believed that the method of the present invention can be applied in various biomolecular studies and pharmacological studies.

It should be understood that the above only illustrates and describes examples whereby the present invention may be carried out, and that modifications and/or alterations may be made thereto without departing from the spirit of the invention.

It should also be understood that certain features or steps of the invention, which are, for clarity, described in the context of separate embodiments, may also be provided in combination in a single embodiment. Conversely, various features or steps of the invention which are, for brevity, described in the context of a single embodiment, may also be provided or separately or in any suitable subcombination.

1. A device for intracellular delivery, comprising a substrate having a diamond layer, and diamond nanoneedles that spaced apart from each other on the diamond layer, the substrate further comprises a silicon layer below the diamond layer, wherein the nanoneedles are cylindrical nanoneedles, and a side surface of the nanoneedles is perpendicular to the diamond layer.

2. The device according to claim 1, wherein the cylindrical nanoneedle has a cross-section with a diameter of 10-800 nm, preferably 50-600 nm, more preferably 200-450 nm; the nanoneedle has a height of 3-8 μm, preferably 3.5-6.5 μm, more preferably 3.8-5.3 μm.

3. The device according to claim 1, wherein the nanoneedles are distributed on the diamond layer with a distribution density of 1×10⁶/cm² to 15×10⁶/cm², preferably 4×10⁶/cm² to 8×10⁶/cm².

4. The device according to claim 1, wherein the diamond layer has a thickness of 0.5-5 μm, the silicon layer has a thickness of 400-600 μm.

5. A method for intracellular delivery, comprising the steps of:
a) depositing a cell in a culture medium on a plate, the culture medium comprises a material to be delivered;
b) providing a device for intracellular delivery on a liquid surface of the culture medium to form a sandwich structure, the device for intracellular delivery includes a substrate and nanoneedles that attached on a substrate surface and spaced apart from each other, the nanoneedles are made from diamond; tips of the nanoneedles point towards the cells;
c) centrifuging the sandwich structure at a centrifugation condition that allows the tips of the nanoneedles to pierce the cells.

6. The method according to claim 5, characterized in that: the device for intracellular delivery is the device according to claim 1.

7. The method according to claim 5, characterized in that: the nanoneedles of the device for intracellular deliver are cone-shaped, and bottom surfaces of the cone-shaped nanoneedles are connected to the diamond layer.

8. The method according to any one of claim 5, characterized in that: the centrifugation condition comprises: the relative centrifugal force is 10-15 g, preferably 12-13 g.

9. The method according to claim 8, characterized in that: the relative centrifugal force of the centrifugation during an acceleration stage has an acceleration rate of 0.001-0.003 g/s; the relative centrifugal force of the centrifugation during a deceleration stage has a deceleration rate of 0.003-0.006 g/s.

10. The method according claim 5, characterized in that: the material to be delivered is selected from the group consisting of: a DNA, a RNA, a PNA, a dye, a protein, an antibody, a small molecule drug, a nanoparticle, and a combination thereof; wherein the dye includes ethidium homodimer and/or a quantum dot, the nanoparticle includes a polyethylene nanoparticle.

11. The method according to claim 10, characterized in that: the material to be delivered is selected from the group consisting of: the DNA, the RNA, the PNA and a combination thereof; the culture medium further includes a nucleic acid transfection reagent, wherein the nucleic acid transfection reagent includes a cationic liposome.

12. A method of preparing a device for intracellular delivery, characterized in that: the device comprise a substrate having a diamond layer, and diamond nanoneedles formed on the diamond layer, the diamond nanoneedles spaced apart from each other, the substrate further comprises a silicon layer below the diamond layer, characterized in that: the nanoneedles are cylindrical nanoneedles, and a side surface of the nanoneedles is perpendicular to the diamond layer; the method further comprises the following steps:
a) forming a nanodiamond film on the silicon layer, a deposition condition allows the nanodiamond film to have a thickness of 0.5-5 μm larger than a desired height of the nanoneedles;
b) performing a bias-assisted reactive ion etching on the nanodiamond film formed, wherein the bias-assisted reactive ion etching is performed under the conditions of: a reactive pressure of 4×10⁻³ to 8×10⁻³ Torr, a reaction time from 20 minutes to 4 hours; a bias pressure of −50V to −250V; and a gas for bias-assisted reactive ion etching is selected from the group consisting: H₂, a mixed gas of Ar and H₂, and a mixed gas of CH₄ and H₂, and a combination thereof.

13. A method for disrupting a cell membrane, comprising the steps of:
providing a centrifuge;
providing a cell incubated in a container with a medium, the medium comprising a liquid surface;
placing a device having nanoneedles on the liquid surface of the medium to form a sandwich structure;
centrifuging the sandwich structure with a centrifugal force;
wherein the nanoneedles are cylindrical, and the centrifugation force is applied with an acceleration rate of from about 0.001 to about 0.003 g/s, and a deceleration rate of about 0.003 to about 0.006 g/s.

14. The method according to claim 13, wherein the centrifugal force applied is a relative centrifugal force of from about 10 to about 15 g.

15. The method according to claim 13, wherein the device is the device according to claim 1.

16. A cell comprising a cell membrane disrupted according to the method of claim 13.
